(19) United States
(12) Patent Application Publication
(10) Pub. No.: US 2014/0088000 A1
(43) Pub. Date: Mar. 27, 2014

(54) Leptin Rescues Neurons from Alzheimer's Disease Related Pathways Triggered by Lipid Burden

(71) Applicant: Neurotez, Inc., Bridgewater, NJ (US)

(72) Inventors: Nikolaos Tezapsidis, West Orange, NJ (US); Steven Grecco, Carlstadt, NJ (US)

(73) Assignee: Neurotez, Inc., Bridgewater, NJ (US)

(21) Appl. No.: 14/039,480

(22) Filed: Sep. 27, 2013

Related U.S. Application Data
(60) Provisional application No. 61/706,550, filed on Sep. 27, 2012.

(51) Int. Cl. A61K 38/22; G01N 33/50

(52) U.S. Cl. CPC A61K 38/2264; G01N 33/5008
USPC 514/5.8; 435/7.92

(57) ABSTRACT

The described invention relates to compositions and methods for rescuing neurons from Alzheimer's Disease related pathways triggered by lipid burden or metabolic insult.

This application claims the benefit of priority of U.S. application 61/706,550, filed Sep. 27, 2012, incorporated herein by reference in its entirety.

### STATEMENT OF GOVERNMENT FUNDING

This invention was made with government support under Grant Number SBIR-1R43AG029670 awarded by the National Institute on Aging. The government has certain rights in the invention.

### FIELD OF THE INVENTION

The described invention relates to compositions and methods for rescuing neurons from Alzheimer's Disease related pathways triggered by lipid burden or metabolic insult.

### BACKGROUND

### Alzheimer's Disease

Alzheimer's disease (also called “AD”, “senile dementia of the Alzheimer Type (SDAT)” or “Alzheimer's”) is a neurodegenerative disorder of the central nervous system (“CNS”). AD is usually diagnosed clinically from the patient history, collateral history from relatives, and clinical observations, based on the presence of characteristic neurological and neuropsychological features.

AD is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyms. Both amyloid plaques (“AP”) and neurofibrillary tangles (“NFT”) are clearly visible by microscopy in brains of those afflicted with AD. Plaques are dense, mostly insoluble deposits of amyloid-beta (“Aβ”) protein and cellular material outside and around neurons. NFT are aggregates of the microtubule-associated protein “tau”, which have become hyperphosphorylated and accumulate inside the cells themselves. Although many older individuals develop some plaques and tangles as a consequence of ageing, the brains of AD patients have a greater number of such plaques and tangles in specific brain regions, such as the temporal lobe.

AD is characterized histologically by the presence of extracellular amyloid deposits in the brain, together with widespread neuronal loss. Extracellular amyloid deposits are known as neuritic or senile plaques. Amyloid deposits also may be found within and around blood vessels. The main protein constituent of AD and AD-like senile plaques is Aβ. Aβ may be detected in plasma and cerebrospinal fluid (“CSF”) in vivo, and in cell culture media in vitro.

The terms “amyloid peptide” “amyloid β peptide” and “Aβ” are used interchangeably herein to refer to the family of peptides generated through proteolytic processing of the amyloid precursor protein (APP).

APP exists as three different spliced isoforms, one having 770 amino acids (isoform a), one having 751 amino acids (isoform b), and one having 695 amino acids. The term “APP” as used herein refers to all three isoforms. The terms “amyloid peptide” “amyloid β peptide” and “Aβ” include, but are not limited to, Aβ40, Aβ42 and Aβ43. The two major forms of Aβ are Aβ40, corresponding to a 40 amino acid-long peptide and Aβ42, corresponding to a 42 amino acid-long peptide. Aβ43 corresponds to a 43 amino acid-long Aβ peptide. The peptide sequences for the APP isoforms and amyloid peptides are disclosed in U.S. Pat. No. 8,227,408 B2.

It generally is believed that brain lipids are intricately involved in Aβ-related pathogenic pathways. The Aβ peptide is the major proteinaceous component of the amyloid plaques found in the brains of AD patients and is regarded by many as the culprit of the disorder. The amount of extracellular Aβ accrued is critical for the pathobiology of AD and depends on the antagonizing rates of its production/secretion and its clearance. Studies have shown that neurons depend on the interaction between Presenilin 1 (“PS1”) and Cytoplasmic-Linker Protein 170 (“CLIP-170”) to both generate Aβ and to take it up through the lipoprotein receptor related protein (“LRP”) pathway. Further to this requirement, formation of Aβ depends on the assembly of key proteins in lipid rafts (“LRs”). The term “lipid rafts” as used herein refers to membrane microdomains enriched in cholesterol, glycosphingolipids and glucosylphosphatidyl-inositol-(GPI)-tagged proteins implicated in signal transduction, protein trafficking and proteolysis. Within the LRs it is believed that Aβ's precursor, Amyloid Precursor Protein (“APP”), a type I membrane protein, is cleaved first by the protease β-secretase (BACE) to generate the C-terminal intermediate fragment of APP, CAPPβ, which remains embedded in the membrane. CAPPβ subsequently is cleaved at a site residing within the lipid bilayer by γ-secretase, a high molecular weight multi-protein complex containing presenilin, (PS1/PS2), nicastrin, PEN-2, and APH-1 or fragments thereof. Aβ finally is released outside the cell, where it may: i) start accumulating following oligomerization and exerting toxicity to neurons, or ii) be removed either by mechanisms of endocytosis (involving apolipoprotein-E (apoE) and LRP or Scavenger Receptors) or by degradation by extracellular proteases including insulin-degrading enzyme (IDE) and neprilysin.

It generally is believed that soluble Aβ oligomers, prior to plaque buildup, exert neurotoxic effects leading to neurodegeneration, synaptic loss and dementia. Further, increased Aβ levels may result from abnormal lipid accumulation, thereby producing altered membrane fluidity and lipid raft composition.

The presence of NFT is a characteristic of AD brains. These aggregations of hyperphosphorylated tau protein also are referred to as “Paired Helical filaments” (PHF). The role of PHF, whether as a primary causative factor in AD or in a more peripheral role, is uncertain. However, the accumulation of PHF cause the destabilization of the microtubule network, thus compromising neuronal scaffolding and disrupting cellular trafficking and signal transduction/communication, and leading to neuronal death.

NFT are not specific to AD; NFT also are seen in Creutzfeldt-Jakob disease, Supranuclear Palsy, corticobasal neurodegeneration and Frontaltemporal Dementia with Parkinsonism linked to chromosome 17 (FTDP-17). This suggests that NFT may represent endpoints leading to neurodegeneration, which may be generated by a number of causative events/insults.

A number of clinical and epidemiological studies have suggested that lifestyle factors, particularly nutrition, mental and physical activities are intricately linked to the etiology of Alzheimer's disease (AD) (1, 2). These studies are supported by the observation that a large percentage of AD patients present with some form of insulin resistance, impaired glucose tolerance or hyperinsulinemia, or are type II diabetic (3). Additionally, a number of traits characteristic of the metabolic syndrome, particularly obesity, dyslipidemia, hypertension, reduced HDL cholesterol and metabolic inflammation, are also AD risk factors (1, 2). Indeed, ‘westernized’ high-caloric diets laden with trans and saturated fatty acids, carbohydrates and cholesterol, along with a sedentary lifestyle, promote brain dysfunction in transgenic animal models of AD (4). These findings illustrate the important connection between caloric regulation and mental health.

Obesity in middle age, particularly central adiposity, has been correlated with increased risk of dementia in later life independent of cardiovascular comorbidities (5, 6). Adipose tissue is the production site of Leptin, a hormone that physiologically functions to regulate lipid storage and mobilization. High concentrations of Leptin receptors have been found in the brain, including within the hippocampus, attesting to the hormone's central as well as peripheral sites of action (7). Direct injection of Leptin into the hippocampus of rodents can improve memory processing and modulate long term potentiation and synaptic plasticity (8). Circulating Leptin is transported into the brain by binding to the lipoprotein receptor megalin at the choroid plexus or via a natural saturable Leptin transporter (9). Obesity in midlife leads to elevated circulating Leptin levels, which can potentially saturate its endogenous transporter across the brain and produce a central Leptin resistance-like state (10). Furthermore, Leptin resistance due to desensitization of signaling pathways or prevention of transport due to high triglycerides has been suggested.

It is speculated that dysregulation of Leptin availability or sensitivity at the hippocampal region over a number of years may contribute to cognitive impairment. For individuals who are obese at midlife, studies have suggested that Leptin's transport efficiency across the blood brain barrier (BBB) is not completely restored even after weight loss by caloric restriction, despite a reduction in circulating Leptin levels (10). Weight loss is frequently observed in AD patients prior to the onset of dementia (11, 12), thus obese individuals may be particularly vulnerable to cognitive dysfunction later in life considering the potential for hypoleptinemia due to adipocyte loss and inefficient transport of Leptin across the BBB. To date a number of reports have addressed the correlation between reduced levels of circulating Leptin and AD risk (13-16), severity of dementia (17) and cognitive decline (18, 19). In particular, a study of 785 dementia-free, older individuals followed for a median of 8.3 years identified those with plasma Leptin levels in the lowest quartile of the study as being at four times greater risk for developing AD than those in the highest quartile (20).

Lipids have been reported to play an important role in activating AD-related pathways (21-23). In cell culture models, excess levels of cholesterol (22, 24), ceramide (25, 26) and oleic acid (23, 27) have been shown to stimulate both Aβ production and hyperphosphorylation of tau. Further, hypercholesterolemia has been reported as an AD risk factor (21, 28) and genetic studies indicate that carriers of one copy of the APOE4 gene, involved in lipid metabolism, are three- to four-fold more likely to develop AD than APOE3 carriers (29). It is unknown whether increased AD susceptibility for 84 carriers is due in part to an impaired ability of the lipoprotein to transport lipids and cholesterol across the BBB into the blood, or solely that its function as a transporter and scavenger of Aβ is compromised, as has been reported (30).

### Leptin

Leptin is a helical protein secreted by adipose tissue, which acts on a receptor site in the ventromedial nucleus of the hypothalamus to curb appetite and increase energy expenditure as body fat stores increase. Leptin levels are 40% higher in women, and show a further 50% rise just before menarche, later returning to baseline levels. Leptin levels are lowered by fasting and increased by inflammation.

Human genes encoding both leptin and the leptin receptor site have been identified. Laboratory mice having mutations on the ob gene, which encodes leptin, become morbidly obese, diabetic, and infertile; administration of leptin to these mice improves glucose tolerance, increases physical activity, reduces body weight by 30%, and restores fertility. Mice with mutations of the db gene, which encodes the leptin receptor, also become obese and diabetic but do not improve with administration of leptin. Although mutations in both the leptin and leptin receptor genes have been found in a small number of morbidly obese human subjects with abnormal eating behavior, the majority of obese persons do not show such mutations, and have normal or elevated circulating levels of leptin. The immune deficiency seen in starvation may result from diminished leptin secretion. Mice lacking the gene for leptin or its receptor show impairment of T-cell function, and, in laboratory studies, leptin has induced a proliferative response in human CD4 lymphocytes.

Leptin also controls insulin sensitivity. Within the CNS, leptin crosses the blood brain barrier to bind specific receptors in the brain to mediate food intake, body weight and energy expenditure. In general, (i) leptin circulates at levels proportional to body fat; (ii) leptin enters the CNS in proportion to its plasma concentration; (iii) leptin receptors are found in brain neurons involved in regulating energy intake and expenditure; and (iv) leptin controls food intake and energy expenditure by acting on receptors in the mediobasal hypothalmus.

It generally is believed that leptin inhibits the activity of neurons that contain neuropeptide Y (NPY) and agouti-related peptide (AgRP), and increases the activity of neurons expressing α-melanocyte-stimulating hormone (α-MSH). The NPY neurons are a key element in the regulation of appetite; small doses of NPY injected into the brains of experimental animals stimulates feeding, while selective destruction of the NPY neurons in mice causes them to become anorexic. Conversely, α-MSH is an important mediator of satiety, and differences in the gene for the receptor at which α-MSH acts in the brain are linked to obesity in humans.

### Leptin and AMPK

Several pieces of evidence suggest that brain metabolic disturbances may precede the pathological cascades characteristic of AD. For example, functional neuroimaging studies, including 2-deoxy-2[(18)F]fluoro-D-glucose (FDG) positron emission topography (PET), have illustrated regional hypometabolism in the early AD brain; and that the pattern correlates with typical brain atrophy in AD (Kapogiannis, D. et al., Lancet Neurol., (2011), 10: 187-198; Sperling R. et al., Neuromolecular Med., (2010), 12: 27-43; Fouquet M. et al., Brain, (2009), 2058-2067; Mosconi L. et al., J. Nucl. Med. Mol. Imaging, (2009), 36: 811-822). Interestingly, pyramidal neurons of the hippocampus have particularly demanding energy needs, rendering the hippocampus a region more sensitive to states of metabolic distress (LaManna, J. et al., Brain Res., (1985), 326: 299-305).

Leptin reduces tau phosphorylation and Aβ production in neuronal cells and transgenic mice models of AD. Leptin's effects in vitro were dependent on activation of the cellular energy sensor, AMP-activated protein kinase (AMPK) (Greco, S. et al., Biochem. Biophys. Res. Commun. (2008), 376: 536-541). AMPK is ubiquitously expressed throughout the body and is activated in states of low cellular energy by an elevated AMP/ATP ratio (Winder, W. et al., Am. J. Physiol. (1999), 277: E1-10). Besides ATP, the only other small molecule in cells that indicates energy status is NAD+, which is necessary for activation of a family of evolutionarily conserved energy sensors, the Sirtuins (SIRT) (Imai, S. et al., Nature (2000), 403: 795-800).

### Sirtuins (SIRT)

The Sirtuins are histone deacetylases that play important roles in a number of physiological processes, including stress resistance (Cohen, H. et al., Science (2004), 305: 390-392), replicative senescence (Chua, K. et al., Cell Metabolism, (2005), 2: 67-76), aging and differentiation (Blander, G. et al., Annual Review of Biochemistry, (2004), 73: 417-435). Notably SIRT1 has been associated with the anti-aging effects of caloric restriction and, most recently, inhibition of amyloidogenic pathways in laboratory models of AD (Chen, D. et al., Science, (2005), 310: 1641; Donmez, G. et al., Cell, (2010), 142: 320-332; Bonda, D. et al., Lancet Neurology (2011), 10: 275-279). Additionally, caloric restriction has been shown to indirectly activate SIRT1 through a linear pathway involving AMPK (Fulco, M. et al., Developmental Cell, (2008), 14: 661-673).

While a high density of functional Leptin receptors have been reported to be expressed in the hippocampus and other cortical regions of the brain, the physiological significance has not been explored extensively.

### SUMMARY OF THE INVENTION

According to one aspect, the described invention provides a method for rescuing neurons from Alzheimer's Disease related pathways triggered by lipid burden, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to rescue neurons from Alzheimer's Disease related pathways triggered by lipid burden or metabolic insult.

As used herein, the term “lipid burden” refers to an excess level of lipid(s), cholesterol, or any combination thereof.

In one aspect, the described invention provides a method to inhibit decreases in cell viability triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit decreases in cell viability triggered by lipid burden or metabolic insult.

In one aspect, the described invention provides a method inhibiting decreases in enzymatic activity of AMP-activated protein kinase (AMPK) triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit the decrease in enzymatic activity of AMP-activated protein kinase (AMPK) in the neuronal cell population triggered by lipid burden or metabolic insult.

In one aspect, the described invention provides a method for inhibiting decreases in enzymatic activity of at least one family member of Sirtuins (SIRT) triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit decreases in enzymatic activity of at least one family member of Sirtuins (SIRT) in the neuronal cell population triggered by lipid burden or metabolic insult.

In one aspect, the described invention provides a method for decreasing the phosphorylation of tau at sites known to be hyperphosphorylated in AD triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit the phosphorylation of tau at sites known to be hyperphosphorylated in AD in the neuronal cell population triggered by lipid burden or metabolic insult.

In one aspect, the described invention provides a method for decreasing the accumulation of Aβ triggered by lipid burden or metabolic in a neuronal cell population insult, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit the accumulation of Aβ in the neuronal cell population triggered by lipid burden or metabolic insult.

In one aspect, the described invention provides in vitro assay methods to screen for compounds that prevent the activation of AD-related pathways mediated by lipid burden or metabolic insult.

### BRIEF DESCRIPTION OF THE DRAWINGS

FIG. 1 shows the ability of Leptin to improve cell viability of RA-SY5Y cells during metabolic insult. Cells were treated for 18 h with ceramide (25 μM), cholesterol (27.5 μg/mL), oleic acid (30 μg/mL) or control (0.125% DMSO or 675 μg/mL MβCD) in the presence or absence of Leptin (10 or 100 ng/mL) and cell viability was measured. Viability was normalized to non-treated cells which were assigned a value of 100%, n=10. *p<0.05 vs. control without metabolic insult. *p<0.05 vs. metabolic insult without Leptin.

FIG. 2 shows the ability of Leptin to enhance AMPK and SIRT activity in RA-SY5Y cells during metabolic insult. Cells were treated for 6 h with ceramide, cholesterol, oleic acid or control in the presence or absence of Leptin (100 ng/mL) and (A) AMPK or (B) SIRT activity measured. All activity values were normalized to total protein, n=3. *p<0.05 vs. control without metabolic insult. *p<0.05 vs. metabolic insult without Leptin.

FIG. 3 shows the ability of Leptin to reverse hyperphosphorylation of tau in RA-SY5Y cells during metabolic insult. Cells were treated for 6 h with ceramide, cholesterol, oleic acid or control in the presence or absence of Leptin (100 ng/mL) and (A) pTau²³¹, (B) pTau³⁹⁶ or total tau measured by ELISA. All concentration values were normalized to total tau, n=3. *p<0.05 vs. control without metabolic insult. *p<0.05 vs. metabolic insult without Leptin.

FIG. 4 shows the ability of Leptin to reverse metabolic insult-induced accumulation of extracellular Aβ₍₁₋₄₀₎ in SY5Y_{APP-751} cells. Cells were treated for 18 h with ceramide, cholesterol, oleic acid or control in the presence or absence of Leptin (100 ng/mL), and culture media collected for determination of AP₍₁₋₄₀₎ levels by ELISA. Results were normalized to total protein and presented as a percentage relative to control, which was assigned a value of 100%, n=3. *p<0.05 vs. control without metabolic insult. *p<0.05 vs. metabolic insult without Leptin.

FIG. 5 shows the effect of various metabolic insults on the viability of RA-SY5Y cell populations. Cells were treated for 18 h with a range of doses of {A) ceramide, (B) cholesterol, (C) oleic acid or vehicle (DMSO or MβCD) and cell viability was measured. Viability was calculated by a standard curve using fixed numbers of cells and normalized to values for non-treated cells—defined as 100% viability, n=10.

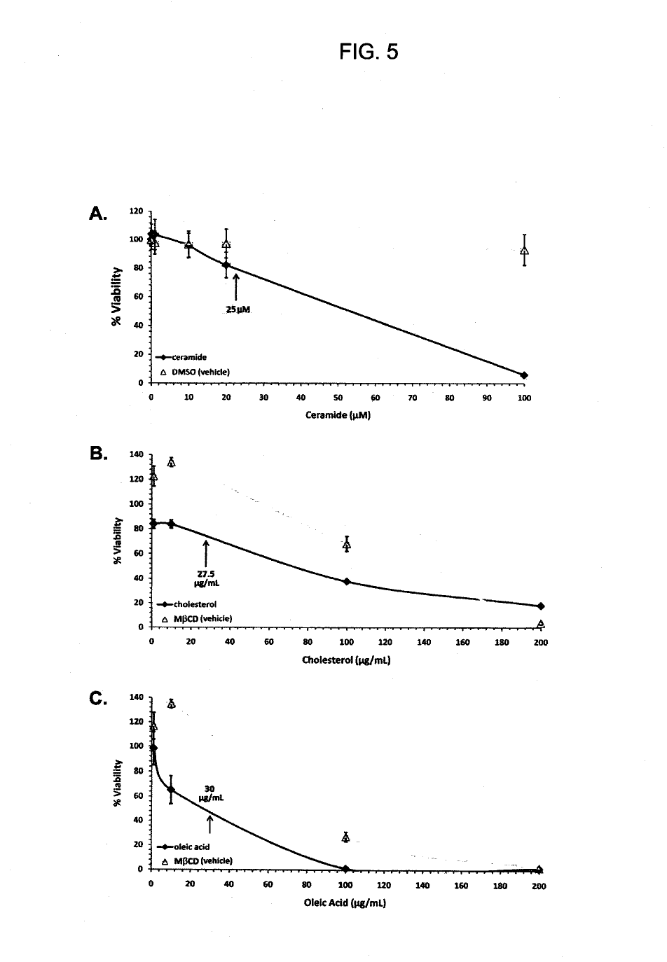

FIG. 6 shoes a flowchart depicting in vitro screening of compounds which prevent activation of AD biochemical pathways in response to metabolic challenges. In order of decreasing throughput and number compounds investigated, dosing studies are first performed in RA-SY5Y to determine feasibility in inhibiting cholesterol-induced cell death. Next, candidate compounds are tested at an effective dose from the first screen in preventing a cholesterol-induced decrease in cellular metabolism through boosting AMPK and SIRT activity. Candidates passing the preliminary screens are then tested in AD-specific assays. First, compounds are tested for their ability to prevent oleic acid-induced hyperphosphorylation of tau in RA-SY5Y cells, and second, SY5Y stably expressing human APP751 (SY5Y_{APP571}) are used for testing any remaining compounds' ability to reduce ceramide-induced accumulation of extracellular Aβ₍₁₋₄₀₎. Compounds which make it through the complete platform are considered leads to be tested in in vivo models. Leptin is utilized throughout the screen as a reference standard.

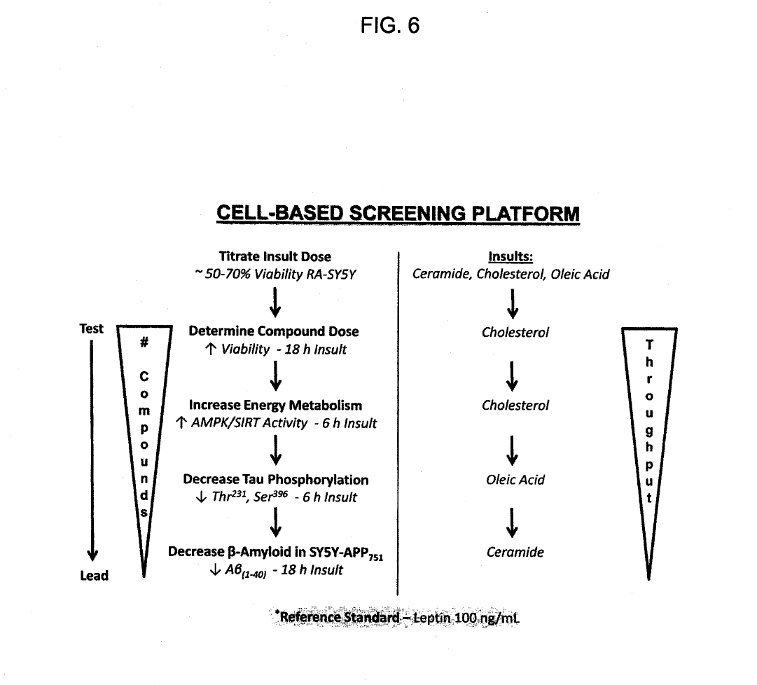

### DETAILED DESCRIPTION OF THE INVENTION

The described invention relates to compositions and methods for rescuing neurons from Alzheimer's Disease related pathways triggered by lipid burden or metabolic insult.

According to one aspect, the described invention provides a method for rescuing neurons from Alzheimer's Disease related pathways triggered by lipid burden or metabolic insult, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to rescue neurons from Alzheimer's Disease related pathways triggered by lipid burden or metabolic insult.

In one aspect, the described invention provides a method to inhibit decreases in cell viability triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit decreases in cell viability triggered by lipid burden or metabolic insult.

In one aspect, the described invention provides a method inhibiting decreases in enzymatic activity of AMP-activated protein kinase (AMPK) triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit the decrease in enzymatic activity of AMP-activated protein kinase (AMPK) in the neuronal cell population triggered by lipid burden or metabolic insult.

In one aspect, the described invention provides a method for inhibiting decreases in enzymatic activity of at least one family member of Sirtuins (SIRT) triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit decreases in enzymatic activity of at least one family member of Sirtuins (SIRT) in the neuronal cell population triggered by lipid burden or metabolic insult.

In one aspect, the described invention provides a method for decreasing the phosphorylation of tau at sites known to be hyperphosphorylated in AD triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit the phosphorylation of tau at sites known to be hyperphosphorylated in AD in the neuronal cell population triggered by lipid burden or metabolic insult.

In one aspect, the described invention provides a method for decreasing the accumulation of Aβ triggered by lipid burden or metabolic insult in a neuronal cell population, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to inhibit the accumulation of Aβ in the neuronal cell population triggered by lipid burden or metabolic insult.

In one aspect, the described invention provides in vitro assay methods to screen for compounds that prevent the activation of AD-related pathways mediated by lipid burden or metabolic insult.

According to one embodiment, the leptin composition comprises a leptin, or a pharmaceutically acceptable salt thereof. According to another embodiment, the leptin composition comprises a leptin mimic, or a pharmaceutically acceptable salt thereof. According to another embodiment, the leptin composition comprises a leptin derivative, or a pharmaceutically acceptable salt thereof. According to another embodiment, the leptin composition comprises a leptin agonist, or a pharmaceutically acceptable salt thereof. According to another embodiment, the leptin composition comprises an AMP-dependent protein kinase activator, or a pharmaceutically acceptable salt thereof. According to another embodiment, the leptin composition comprises a mimic of a leptin blocker, or a pharmaceutically acceptable salt thereof. According to another embodiment, the leptin composition comprises a leptin antagonist, or a pharmaceutically acceptable salt thereof. According to another embodiment, the leptin composition comprises an AMP-dependent protein kinase inhibitor, or a pharmaceutically acceptable salt thereof.

According to another embodiment, the leptin composition comprises at least one of a leptin, a leptin mimic, a leptin derivative, a leptin agonist, an AMP-dependent protein kinase activator, a mimic of a leptin blocker, a leptin antagonist, an AMP-dependent protein kinase inhibitor, or pharmaceutically acceptable salts thereof.

The term “treat” or “treating” as used herein refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

The term “reduce” or “reducing” as used herein refers to limit occurrence of the disorder in individuals at risk of developing the disorder.

The term “modulate” as used herein means to regulate, alter, adapt, or adjust to a certain measure or proportion.

The term “disease” or “disorder” as used herein refers to an impairment of health or a condition of abnormal functioning. The term “syndrome,” as used herein, refers to a pattern of symptoms indicative of some disease or condition. The term “injury,” as used herein, refers to damage or harm to a structure or function of the body caused by an outside agent or force, which may be physical or chemical. The term “condition”, as used herein, refers to a variety of health states and is meant to include disorders or diseases caused by any underlying mechanism or disorder, injury, and the promotion of healthy tissues and organs.

The term “administering” as used herein refers to causing to take or apportioning and includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The terms “subject” or “individual” or “patient” are used interchangeably to refer to a member of an animal species of mammalian origin, including humans.

The term “peptidomimetic” refers to a small protein-like chain designed to mimic or imitate a peptide. A peptidomimetic may comprise non-peptidic structural elements capable of mimicking (meaning imitating) or antagonizing (meaning neutralizing or counteracting) the biological action(s) of a natural parent peptide. The terms “leptin peptidomimetic” “leptin mimic”, and “leptin mimetic” are used interchangeably herein to refer to a leptin derivative comprising a functional domain of a leptin protein that produces a biological effect. In chemistry, a derivative is a compound that at least theoretically may be formed from a precursor compound. These derivatives may be combined with another molecule to produce or enhance the biological effect. The biological effect may include, for example, but is not limited to, modulating amyloid peptide levels within a subject; modulating tau phosphorylation levels within a subject; decreasing amyloid peptide levels within a subject; decreasing tau phosphorylation levels within a subject, and the like.

The term “antagonist” as used herein refers to a substance that counteracts the effects of another substance. The term “agonist” as used herein refers to a chemical substance capable of activating a receptor to induce a full or partial pharmacological response. The term “blocker” as used herein refers to a substance that inhibits the physiological action of another substance.

The term “leptin agonist” refers to a compound capable of activating the leptin receptor and/or downstream effectors and of modulating amyloid peptide levels or tau phosphorylation in a subject. Such effectors may include, for example, but are not limited to, AMP-dependent protein kinase (“AMPK”) and sterol regulatory element binding proteins (“SREBP”).

The leptin receptor (OB-R), a member of the class I cytokine receptor superfamily, has at least six isoforms as a result of alternative splicing. As used herein the term “isoform” refers to a version of a protein that has the same function as another protein but that has some small difference(s) in its sequence. All isoforms of OB-R share an identical extracellular ligand-binding domain. Leptin's functional receptor (OB-Rb), the b isoform, is expressed not only in the hypothalamus, where it regulates energy homeostasis and neuroendocrine function, but also in other brain regions and in the periphery, including all cell types of innate and adaptive immunity. The full-length b isoform (OB-Rb) lacks intrinsic tyrosine kinase activity and is involved in several downstream signal transduction pathways.

The terms “therapeutically effective amount”, an “amount effective”, or “pharmaceutically effective amount” of one or more of the active agents are used interchangeably to refer to an amount that is sufficient to provide the intended benefit of treatment. An effective amount of the active agents that can be employed according to the described invention generally ranges from generally about 0.01 mg/kg body weight to about 100 g/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition resulting from accumulation of an amyloid peptide in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition.

### Methods for Inhibiting Decreases in Cell Viability in a Neuronal Cell Population Triggered by Lipid Burden or Metabolic Insult

According to one aspect, the described invention provides a method for inhibiting decreases in in cell viability triggered by lipid burden or metabolic insult in a neuronal cell population, comprising contacting the neuronal cell population with a composition containing an effective amount of Leptin, a Leptin analog or derivative, or a Leptin agonist, and a carrier.

According to some embodiments, the Leptin analog or derivative includes functional analogs of Leptin, which are capable of binding to a Leptin receptor (OB-R) and are able to induce a signal transduction pathway via the Leptin receptor inside the cell. Examples of the Leptin analog or derivative include, but are not limited to, adiponectin (such as, human, mouse, and rat adiponectin), LY396623, Metreleptin, a murine Leptin analog, pegylated Leptin, and methionyl human Leptin, and Resistin (such as human, mouse, and rat Resistin).

According to some other embodiments, the Leptin analog or derivative includes a peptide or polypeptide in which at least one amino acid residue has been replaced with non-naturally occurring amino acids, including, but not limited to, beta-alanine, alpha amino butyric acid, gamma-amino butyric acid, alpha-isobutryic acid, norvaline, norleucine, epsilon-lysine, ornithine, homoserine, and hydroxyproline.

According to some embodiments, the Leptin agonist includes a compound capable of activating the Leptin receptor and/or its downstream effectors, such as AMP-activated protein kinase (AMPK), inside a cell. According to some such embodiments, the Leptin agonist comprises phenformin, 5-aminoimidazole-4-carboxamide riboside (AICAR), metformin, rosiglitazone, or a combination thereof.

According to one embodiment of the method, the composition is administered to a mammal in vivo. According to another embodiment, the composition is administered ex vivo.

According to another embodiment, the composition is administered systemically, for example, orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means, such as, but not limited to, injection, implantation, grafting, or topical application. The term “topical” as used herein refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

According to another embodiment, the composition is administered parenterally. The term “parenteral” as used herein refers to introduction into the body by way of an injection (i.e., administration by injection), including, for example, subcutaneous injection (i.e., an injection beneath the skin), intramuscular injection (i.e., an injection into a muscle), intravenous injection (i.e., an injection into a vein), intrathecal injection (i.e., an injection into the space around the spinal cord or under the arachnoid membrane of the brain), intrasternal injection (i.e., injection into the sternum (a long flat bone that is situated along the ventral midline of the thorax and articulates with the ribs)), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

According to another embodiment, administering can be performed once, a plurality of times, and/or over one or more extended periods either as individual unit doses or in the form of a treatment regimen comprising multiple unit doses of multiple drugs and/or substances.

According to another embodiment, the neuronal cell population comprises a neuronal population of the central nervous system, which expresses a Leptin receptor (OB-R). According to another embodiment, the receptor is Obese Receptor-Rb (Ob-Rb).

According to another embodiment, the neuronal cell population includes, but is not limited to, a population of RA-SY5Y cells, a hippocampal neuron population, a cortical neuron population, a Purkinje neuron population, a basal ganglia neuron population, an olfactory neuron population, a dopaminergic neuron population, a noradrenergic neuron population, or a combination thereof. According to another embodiment, the neuronal cell population comprises a motor neuron population. According to another embodiment, the motor neuron population comprises a spinal motor neuron population. According to another embodiment, the neuronal cell population comprises an interneuron population. According to another embodiment, the neuronal cell population comprises a neuron population of the peripheral nervous system.

### Methods for Inhibiting Decreases in Enzymatic Activity of at Least One Family Member of Sirtuins (SIRT) in a Neuronal Cell Population Triggered by Lipid Burden or Metabolic Insult

According to one aspect, the described invention provides a method for inhibiting decreases in enzymatic activity of at least one family member of Sirtuins (SIRT) triggered by lipid burden or metabolic insult in a neuronal cell population, comprising contacting the neuronal cell population with a composition containing an effective amount of Leptin, a Leptin analog or derivative, or a Leptin agonist, and a carrier.

According to some embodiments, the Leptin analog or derivative includes functional analogs of Leptin, which are capable of binding to a Leptin receptor (OB-R) and are able to induce a signal transduction pathway via the Leptin receptor inside the cell. Examples of the Leptin analog or derivative include, but are not limited to, adiponectin (such as, human, mouse, and rat adiponectin), LY396623, Metreleptin, a murine Leptin analog, pegylated Leptin, and methionyl human Leptin, and Resistin (such as human, mouse, and rat Resistin).

According to some other embodiments, the Leptin analog or derivative includes a peptide or polypeptide in which at least one amino acid residue has been replaced with non-naturally occurring amino acids, including, but not limited to, beta-alanine, alpha amino butyric acid, gamma-amino butyric acid, alpha-isobutryic acid, norvaline, norleucine, epsilon-lysine, ornithine, homoserine, and hydroxyproline.

According to some embodiments, the Leptin agonist includes a compound capable of activating the Leptin receptor and/or its downstream effectors, such as AMP-activated protein kinase (AMPK), inside a cell. According to some such embodiments, the Leptin agonist comprises phenformin, 5-aminoimidazole-4-carboxamide riboside (AICAR), metformin, rosiglitazone, or a combination thereof.

According to one embodiment of the method, the composition is administered to a mammal in vivo. According to another embodiment, the composition is administered ex vivo.

According to another embodiment, the composition is administered systemically, for example, orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means, such as, but not limited to, injection, implantation, grafting, or topical application. The term “topical” as used herein refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

According to another embodiment, the composition is administered parenterally. The term “parenteral” as used herein refers to introduction into the body by way of an injection (i.e., administration by injection), including, for example, subcutaneous injection (i.e., an injection beneath the skin), intramuscular injection (i.e., an injection into a muscle), intravenous injection (i.e., an injection into a vein), intrathecal injection (i.e., an injection into the space around the spinal cord or under the arachnoid membrane of the brain), intrasternal injection (i.e., injection into the sternum (a long flat bone that is situated along the ventral midline of the thorax and articulates with the ribs)), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

According to another embodiment, administering can be performed once, a plurality of times, and/or over one or more extended periods either as individual unit doses or in the form of a treatment regimen comprising multiple unit doses of multiple drugs and/or substances.

According to another embodiment, the family member of Sirtuin is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

According to another embodiment, the enzymatic activity of total Sirtuins in the neuronal cell population treated with the composition is at least two times greater than the enzymatic activity of total Sirtuins in a control neuron population without treatment.

According to another embodiment, the neuronal cell population comprises a neuronal population of the central nervous system, which expresses a Leptin receptor (OB-R). According to another embodiment, the receptor is Obese Receptor-Rb (Ob-Rb).

According to another embodiment, the neuronal cell population includes, but is not limited to, a population of RA-SY5Y cells, a hippocampal neuron population, a cortical neuron population, a Purkinje neuron population, a basal ganglia neuron population, an olfactory neuron population, a dopaminergic neuron population, a noradrenergic neuron population, or a combination thereof. According to another embodiment, the neuronal cell population comprises a motor neuron population. According to another embodiment, the motor neuron population comprises a spinal motor neuron population. According to another embodiment, the neuronal cell population comprises an interneuron population. According to another embodiment, the neuronal cell population comprises a neuron population of the peripheral nervous system.

### Methods for Decreasing the Phosphorylation of Tau at Sites Known to be Hyperphosphorylated in AD in a Neuronal Cell Population Triggered by Lipid Burden or Metabolic Insult

According to one aspect, the described invention provides a method for decreasing the phosphorylation of tau at sites known to be hyperphosphorylated in AD triggered by lipid burden or metabolic insult in a neuronal cell population, comprising contacting the neuronal cell population with a composition containing an effective amount of Leptin, a Leptin analog or derivative, or a Leptin agonist, and a carrier.

In one embodiment, the phosphorylation of tau at sites known to be hyperphosphorylated in AD is at least one amino acid residue selected from the group consisting of Ser-202/Thr-205 (AT8 site), Ser-214, Ser-181, Ser-212 (AT100 site), Thr-231, Ser-235 (TG3 site), and Ser-326/Ser-404 (PHF-1 site).

According to some embodiments, the Leptin analog or derivative includes functional analogs of Leptin, which are capable of binding to a Leptin receptor (OB-R) and are able to induce a signal transduction pathway via the Leptin receptor inside the cell. Examples of the Leptin analog or derivative include, but are not limited to, adiponectin (such as, human, mouse, and rat adiponectin), LY396623, Metreleptin, a murine Leptin analog, pegylated Leptin, and methionyl human Leptin, and Resistin (such as human, mouse, and rat Resistin).

According to some other embodiments, the Leptin analog or derivative includes a peptide or polypeptide in which at least one amino acid residue has been replaced with non-naturally occurring amino acids, including, but not limited to, beta-alanine, alpha amino butyric acid, gamma-amino butyric acid, alpha-isobutryic acid, norvaline, norleucine, epsilon-lysine, ornithine, homoserine, and hydroxyproline.

According to some embodiments, the Leptin agonist includes a compound capable of activating the Leptin receptor and/or its downstream effectors, such as AMP-activated protein kinase (AMPK), inside a cell. According to some such embodiments, the Leptin agonist comprises phenformin, 5-aminoimidazole-4-carboxamide riboside (AICAR), metformin, rosiglitazone, or a combination thereof.

According to one embodiment of the method, the composition is administered to a mammal in vivo. According to another embodiment, the composition is administered ex vivo.

According to another embodiment, the composition is administered systemically, for example, orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means, such as, but not limited to, injection, implantation, grafting, or topical application. The term “topical” as used herein refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

According to another embodiment, the composition is administered parenterally. The term “parenteral” as used herein refers to introduction into the body by way of an injection (i.e., administration by injection), including, for example, subcutaneous injection (i.e., an injection beneath the skin), intramuscular injection (i.e., an injection into a muscle), intravenous injection (i.e., an injection into a vein), intrathecal injection (i.e., an injection into the space around the spinal cord or under the arachnoid membrane of the brain), intrasternal injection (i.e., injection into the sternum (a long flat bone that is situated along the ventral midline of the thorax and articulates with the ribs)), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

According to another embodiment, administering can be performed once, a plurality of times, and/or over one or more extended periods either as individual unit doses or in the form of a treatment regimen comprising multiple unit doses of multiple drugs and/or substances.

According to another embodiment, the family member of Sirtuin is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

According to another embodiment, the enzymatic activity of total Sirtuins in the neuronal cell population treated with the composition is at least two times greater than the enzymatic activity of total Sirtuins in a control neuron population without treatment.

According to another embodiment, the neuronal cell population comprises a neuronal population of the central nervous system, which expresses a Leptin receptor (OB-R). According to another embodiment, the receptor is Obese Receptor-Rb (Ob-Rb).

According to another embodiment, the neuronal cell population includes, but is not limited to, a population of RA-SY5Y cells, a hippocampal neuron population, a cortical neuron population, a Purkinje neuron population, a basal ganglia neuron population, an olfactory neuron population, a dopaminergic neuron population, a noradrenergic neuron population, or a combination thereof. According to another embodiment, the neuronal cell population comprises a motor neuron population. According to another embodiment, the motor neuron population comprises a spinal motor neuron population. According to another embodiment, the neuronal cell population comprises an interneuron population. According to another embodiment, the neuronal cell population comprises a neuron population of the peripheral nervous system.

### Methods for Decreasing the Accumulation of Aβ in a Neuronal Cell Population Triggered by Lipid Burden or Metabolic Insult

According to one aspect, the described invention provides a method for decreasing the accumulation of Aβ triggered by lipid burden or metabolic insult in a neuronal cell population, comprising contacting the neuronal cell population with a composition containing an effective amount of Leptin, a Leptin analog or derivative, or a Leptin agonist, and a carrier.

According to some embodiments, the Leptin analog or derivative includes functional analogs of Leptin, which are capable of binding to a Leptin receptor (OB-R) and are able to induce a signal transduction pathway via the Leptin receptor inside the cell. Examples of the Leptin analog or derivative include, but are not limited to, adiponectin (such as, human, mouse, and rat adiponectin), LY396623, Metreleptin, a murine Leptin analog, pegylated Leptin, and methionyl human Leptin, and Resistin (such as human, mouse, and rat Resistin).

According to some other embodiments, the Leptin analog or derivative includes a peptide or polypeptide in which at least one amino acid residue has been replaced with non-naturally occurring amino acids, including, but not limited to, beta-alanine, alpha amino butyric acid, gamma-amino butyric acid, alpha-isobutryic acid, norvaline, norleucine, epsilon-lysine, ornithine, homoserine, and hydroxyproline.

According to some embodiments, the Leptin agonist includes a compound capable of activating the Leptin receptor and/or its downstream effectors, such as AMP-activated protein kinase (AMPK), inside a cell. According to some such embodiments, the Leptin agonist comprises phenformin, 5-aminoimidazole-4-carboxamide riboside (AICAR), metformin, rosiglitazone, or a combination thereof.

According to one embodiment of the method, the composition is administered to a mammal in vivo. According to another embodiment, the composition is administered ex vivo.

According to another embodiment, the composition is administered systemically, for example, orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means, such as, but not limited to, injection, implantation, grafting, or topical application. The term “topical” as used herein refers to administration of a composition at, or immediately beneath, the point of application. The phrase “topically applying” describes application onto one or more surfaces(s) including epithelial surfaces. Topical administration, in contrast to transdermal administration, generally provides a local rather than a systemic effect.

According to another embodiment, the composition is administered parenterally. The term “parenteral” as used herein refers to introduction into the body by way of an injection (i.e., administration by injection), including, for example, subcutaneous injection (i.e., an injection beneath the skin), intramuscular injection (i.e., an injection into a muscle), intravenous injection (i.e., an injection into a vein), intrathecal injection (i.e., an injection into the space around the spinal cord or under the arachnoid membrane of the brain), intrasternal injection (i.e., injection into the sternum (a long flat bone that is situated along the ventral midline of the thorax and articulates with the ribs)), or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

According to another embodiment, administering can be performed once, a plurality of times, and/or over one or more extended periods either as individual unit doses or in the form of a treatment regimen comprising multiple unit doses of multiple drugs and/or substances.

According to another embodiment, the family member of Sirtuin is selected from the group consisting of Sirtuin-1 (SIRT1), Sirtuin-2 (SIRT2), Sirtuin-3 (SIRT3), Sirtuin-4 (SIRT4), Sirtuin-5 (SIRT 5), Sirtuin-6 (SIRT6), Sirtuin-7 (SIRT7), and a combination thereof.

According to another embodiment, the enzymatic activity of total Sirtuins in the neuronal cell population treated with the composition is at least two times greater than the enzymatic activity of total Sirtuins in a control neuron population without treatment.

According to another embodiment, the neuronal cell population comprises a neuronal population of the central nervous system, which expresses a Leptin receptor (OB-R). According to another embodiment, the receptor is Obese Receptor-Rb (Ob-Rb).

According to another embodiment, the neuronal cell population includes, but is not limited to, a population of RA-SY5Y cells, a hippocampal neuron population, a cortical neuron population, a Purkinje neuron population, a basal ganglia neuron population, an olfactory neuron population, a dopaminergic neuron population, a noradrenergic neuron population, or a combination thereof. According to another embodiment, the neuronal cell population comprises a motor neuron population. According to another embodiment, the motor neuron population comprises a spinal motor neuron population. According to another embodiment, the neuronal cell population comprises an interneuron population. According to another embodiment, the neuronal cell population comprises a neuron population of the peripheral nervous system.

For any Leptin, Leptin analogs or derivatives, or Leptin agonists, described herein, the therapeutically effective amount may be initially determined from preliminary in vitro studies and/or animal models. A therapeutically effective dose also may be determined from human data for therapeutic agent(s) which have been tested in humans and for compounds which are known to exhibit similar pharmacological activities, such as other related active agents. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

According to another embodiment, the therapeutically effective amount of the composition is of an amount from about 0.000001 mg/kg body weight to about 10 g/kg body weight. According to another embodiment, the therapeutically effective amount of the composition is of an amount from about 0.000002 mg/kg body weight to about 10 g/kg body weight. According to another embodiment, the therapeutically effective amount of the composition is of an amount from about 0.000003 mg/kg body weight to about 10 g/kg body weight. According to another embodiment, the therapeutically effective amount of the composition is of an amount from about 0.000004 mg/kg body weight to about 10 g/kg body weight. According to another embodiment, the therapeutically effective amount of the composition is of an amount from about 0.000005 mg/kg body weight to about 10 g/kg body weight. According to another embodiment, the therapeutically effective amount of the composition is of an amount from about 0.000006 mg/kg body weight to about 10 g/kg body weight. According to another embodiment, the therapeutically effective amount of the composition is of an amount from about 0.000007 mg/kg body weight to about 10 g/kg body weight. According to another embodiment, the therapeutically effective amount of the composition is of an amount from about 0.000008 mg/kg body weight to about 10 g/kg body weight. According to another embodiment, the therapeutically effective amount of the composition is of an amount from about 0.000009 mg/kg body weight to about 10 g/kg body weight. According to another embodiment, the therapeutically effective amount of the composition is of an amount from about 0.00001 mg/kg body weight to about 10 g/kg body weight. According to another embodiment, the therapeutically effective amount of the composition is of an amount from about 0.00002 mg/kg body weight to about 10 g/kg body weight. According to another embodiment, the therapeutically effective amount of the composition is of an amount from about 0.0003 mg/kg body weight to about 10 g/kg body weight. According to another embodiment, the therapeutically effective amount of the composition is of an amount from about 0.00004 mg/kg body weight to about 10 g/kg body weight. According to another embodiment, the therapeutically effective amount of the composition is of an amount from about 0.00005 mg/kg body weight to about 10 g/kg body weight. According to another embodiment, the therapeutically effective amount of the composition is of an amount from about 0.00006 mg/kg body weight to about 10 g/kg body weight. According to another embodiment, the therapeutically effective amount of the composition is of an amount from about 0.00007 mg/kg body weight to about 10 g/kg body weight. According to another embodiment, the therapeutically effective amount of the composition is of an amount from about 0.00008 mg/kg body weight to about 10 g/kg body weight. According to another embodiment, the therapeutically effective amount of the composition is of an amount from about 0.00009 mg/kg body weight to about 10 g/kg body weight. According to another embodiment, the therapeutically effective amount of the composition is of an amount from about 0.0001 mg/kg body weight to about 10 g/kg body weight. According to another embodiment, the therapeutically effective amount of the composition is of an amount of about 0.0005 mg/kg body weight. According to another embodiment, the therapeutically effective amount of the composition is of an amount of about 0.001 mg/kg body weight. According to another embodiment, the therapeutically effective amount of the composition is of an amount of about 0.005 mg/kg body weight. According to another embodiment, the therapeutically effective amount of the composition is of an amount of about 0.01 mg/kg body weight. According to another embodiment, the therapeutically effective amount of the composition is of an amount of about 0.1 mg/kg body weight. According to another embodiment, the therapeutically effective amount of the composition is of an amount of about 1 mg/kg body weight. According to another embodiment, the therapeutically effective amount of the composition is of an amount of about 10 mg/kg body weight. According to another embodiment, the therapeutically effective amount of the composition is of an amount of about 20 mg/kg body weight. According to another embodiment, the therapeutically effective amount of the composition is of an amount of about 30 mg/kg body weight. According to another embodiment, the therapeutically effective amount of the composition is of an amount of about 40 mg/kg body weight. According to another embodiment, the therapeutically effective amount of the composition is of an amount of about 50 mg/kg body weight. According to another embodiment, the therapeutically effective amount of the composition is of an amount of about 60 mg/kg body weight. According to another embodiment, the therapeutically effective amount of the composition is of an amount of about 70 mg/kg body weight. According to another embodiment, the therapeutically effective amount of the composition is of an amount of about 80 mg/kg body weight. According to another embodiment, the therapeutically effective amount of the composition is of an amount of about 90 mg/kg body weight. According to another embodiment, the therapeutically effective amount of the composition is of an amount of about 100 mg/kg body weight. According to another embodiment, the therapeutically effective amount of the composition is of an amount of about 110 mg/kg body weight. According to another embodiment, the therapeutically effective amount of the composition is of an amount of about 120 mg/kg body weight. According to another embodiment, the therapeutically effective amount of the composition is of an amount of about 130 mg/kg body weight. According to another embodiment, the therapeutically effective amount of the composition is of an amount of about 140 mg/kg body weight. According to another embodiment, the therapeutically effective amount of the composition is of an amount of about 150 mg/kg body weight. According to another embodiment, the therapeutically effective amount of the composition is of an amount of about 160 mg/kg body weight. According to another embodiment, the therapeutically effective amount of the composition is of an amount of about 170 mg/kg body weight. According to another embodiment, the therapeutically effective amount of the composition is of an amount of about 180 mg/kg body weight. According to another embodiment, the therapeutically effective amount of the composition is of an amount of about 190 mg/kg body weight. According to another embodiment, the therapeutically effective amount of the composition is of an amount of about 200 mg/kg body weight. According to another embodiment, the therapeutically effective amount of the composition is of an amount of about 250 mg/kg body weight. According to another embodiment, the therapeutically effective amount of the composition is of an amount of about 500 mg/kg body weight.

The composition of the described invention may be presented conveniently in unit dosage form and may be prepared by any of the methods well known in the art of pharmacy. All methods include the step of bringing into association a therapeutic agent(s) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The compositions of the described invention may be administered in pharmaceutically acceptable solutions, which may routinely contain pharmaceutically acceptable concentrations of salt, buffering agents, preservatives, compatible carriers, adjuvants, and optionally other therapeutic ingredients.

According to some embodiments, the compositions of the described invention can further include one or more additional compatible active ingredients. The term “compatible” as used herein means that components of a composition are capable of being combined with each other in a manner such that there is no interaction that would substantially reduce the efficacy of the composition under ordinary use conditions. For example, without limitation, the Leptin, Leptin analogs or derivative or Leptin agonist described herein may be mixed with other active materials that do not impair the desired action, or with materials that supplement the desired action.

Solutions or suspensions used for parenteral, intradermal, subcutaneous, subdural, intracerebral, intrathecal, or topical application may include, but are not limited to, for example, the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules (or ampules), disposable syringes or multiple dose vials made of glass or plastic. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

The sterile injectable preparation may also be a sterile injectable solution or suspension in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran.

In some embodiments, the compositions of the present invention may be formulated with an excipient or carrier including, but not limited to, a solvent. The terms “excipient” or “carrier” refer to substances that do not deleteriously react with the active compounds. Carriers must be of sufficiently high purity and of sufficiently low toxicity to render them suitable for administration to the subject being treated. The carrier can be inert, or it can possess pharmaceutical benefits.

The carrier can be liquid or solid and is selected with the planned manner of administration in mind to provide for the desired bulk, consistency, etc., when combined with an active and the other components of a given composition. Typical pharmaceutical carriers include, but are not limited to, binding agents (including, but not limited to pregelatinized maize starch, polyvinylpyrrolidone or hydroxypropyl methylcellulose); fillers (including but not limited to lactose and other sugars, microcrystalline cellulose, pectin, gelatin, calcium sulfate, ethyl cellulose, polyacrylates or calcium hydrogen phosphate.); lubricants (including, but not limited to magnesium stearate, talc, silica, sollidal silicon dioxide, stearic acid, metallic stearates, hydrogenated vegetable oils, corn starch, polyethylene glycols, sodium benzoate, sodium acetate); disintegrants (including but not limited to starch, sodium starch glycolate) and wetting agents (including but not limited to sodium lauryl sulfate). Additional suitable carriers for the compositions of the present invention include, but are not limited to, water, salt solutions, alcohol, vegetable oils, polyethylene glycols, gelatin, lactose, amylose, magnesium stearate, talc, silicic acid, viscous paraffin, perfume oil; fatty acid monoglycerides and diglycerides, petroethral fatty acid esters, hydroxymethylcellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds.

The term “pharmaceutically acceptable carrier” as used herein refers to any substantially non-toxic carrier conventionally useful for administration of pharmaceuticals in which the active component will remain stable and bioavailable. In some embodiments, the pharmaceutically acceptable carrier of the compositions of the present invention include a release agent such as a sustained release or delayed release carrier. In such embodiments, the carrier can be any material capable of sustained or delayed release of the active ingredient to provide a more efficient administration, resulting in less frequent and/or decreased dosage of the active ingredient, ease of handling, and extended or delayed effects.

A composition of the present invention, alone or in combination with other active ingredients, may be administered to a subject in a single dose or multiple doses over a period of time.

The pharmaceutical effect can be curing, minimizing, preventing or ameliorating a disease or disorder, or may have any other pharmaceutical beneficial effect. The concentration of the substance is selected so as to exert its or pharmaceutical effect, but low enough to avoid significant side effects within the scope and sound judgment of the physician. The effective amount of the composition may vary with the age and physical condition of the biological subject being treated, the severity of the condition, the duration of the treatment, the nature of concurrent therapy, the specific composition or other active ingredient employed, the particular carrier utilized, and like factors.

The concentration of the active in the compositions is selected so as to exert its therapeutic effect, but low enough to avoid significant side effects within the scope and sound judgment of the skilled artisan. The effective amount of the composition may vary with the age and physical condition of the biological subject being treated, the severity of the condition, the duration of the treatment, the nature of concurrent therapy, the specific compound, composition or other active ingredient employed, the particular carrier utilized, and like factors. Those of skill in the art can readily evaluate such factors and, based on this information, determine the particular pharmaceutically effective amount of the compositions.

A skilled artisan can determine a pharmaceutically effective amount of the inventive compositions by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED₅₀. The use of the term “individual” distinguishes the ED₅₀ based on the intensity of effect as used herein from the median effective dose, also abbreviated ED₅₀, determined from frequency of response data in a population. “Efficacy” as used herein refers to the property of the compositions of the present invention to achieve the desired response, and “maximum efficacy” refers to the maximum achievable effect. The amount of the compositions of the present invention that will be effective in the treatment of a particular disorder or condition will depend on the nature of the disorder or condition, and can be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN′S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed also will depend on the route of administration, and the seriousness of the disease or disorder, and should be decided according to the judgment of the practitioner and each patient's circumstances. Various administration patterns will be apparent to those skilled in the art.

Those skilled in the art will recognize that initial indications of the appropriate therapeutic dosage of the compositions of the invention can be determined in in vitro and in vivo animal model systems, and in human clinical trials. One of skill in the art would know to use animal studies and human experience to identify a dosage that can safely be administered without generating toxicity or other side effects. For acute treatment, it is preferred that the therapeutic dosage be close to the maximum tolerated dose. For chronic preventive use, lower dosages may be desirable because of concerns about long term effects. Additional compositions of the present invention can be readily prepared using technology which is known in the art such as described in Remington's Pharmaceutical Sciences, 18th or 19th editions, published by the Mack Publishing Company of Easton, Pa., which is incorporated herein by reference.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the present invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention may be embodied in other specific forms without departing from the spirit or essential attributes thereof and, accordingly, reference should be made to the appended claims, rather than to the foregoing specification, as indicating the scope of the invention.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Summary of Experimental Findings

Adipocyte-derived Leptin is a pleiotropic hormone implicated in control of lipid storage and mobilization, bone homeostasis, immune function and neuronal plasticity. Leptin has been shown to prevent accumulation of extracellular AB and hyperphosphorylation of tau in both cell culture and animal models. Herein an investigation was undertaken to test Leptin's ability to prevent the activation of AD-related pathways in neurons following their exposure to a high concentration of a variety of lipids. Specifically, cholesterol, oleic acid and/or ceramide were added to the media of cells to decrease cellular viability and energy metabolism, and increase tau phosphorylation and extracellular A. Leptin increased viability, boosted cellular metabolism by activating AMP-activated protein kinase (AMPK) and the sirtuins (SIRT) and reduced tau phosphorylation and Aβ accumulation in a dose-dependent manner in response to select challenges. These findings demonstrate that Leptin can attenuate activation of AD pathways in response to metabolic stress, and also provide the basis for a novel screening platform to identify new compounds which behave similarly to Leptin.

### Introduction

Our laboratory has previously demonstrated that Leptin facilitates the uptake of Aβ by apoE (30). Further, we have shown that Leptin reduces the amount of Aβ secreted into the medium in a time- and dose-dependent fashion, which is co-incident with composition changes in lipid rafts and redistribution of β-secretase (BACE) and APP within membranes in neurons leading to suboptimum BACE activity (30). We have also observed that Leptin reduces both tau phosphorylation and Aβ production, and improves cognitive performance in a transgenic model of AD (31). To correlate these findings with the metabolic stresses associated with AD risk, we investigated the ability of Leptin to suppress AD-related pathway activation following lipid challenges in neurons.

### Materials and Methods

Reagents:

Minimum essential medium (MEM) was purchased from ATCC (Manassas, Va.). Trypsin-EDTA and penicillin solution were purchased from MP Biomedicals (Solon, Ohio). Fetal bovine serum (FBS), all-trans retinoic acid (ATRA), 0418, nicotinamide, methyl-β-cyclodextrin (MβCD), water-soluble cholesterol, water-soluble oleic acid and recombinant human Leptin were purchased from Sigma-Aldrich (St. Louis, Mo.). The AMPK inhibitor, Compound C, and C2 ceramide were purchased from EMD Biosciences (San Diego, Calif.).

Culture and Stable Transfection of Cell Lines:

The human neuroblastoma cell line, SH-SY5Y, was purchased from ATCC. Cell culture was performed according to manufacturer's specific guidelines. Cells were propagated in MEM containing 10% FBS. Neuronal differentiation was performed as described previously (32).

To generate SY5Y stably over-expressing amyloid precursor protein (APP), cells were transfected with a mammalian expression vector encoding the 751 amino acid isoform of human APP (APP751—Accession #NM 201413) (Origene Technologies; Rockville, Md.) using the FuOENE HD transfection reagent, according to manufacturer's specific instructions (Promega; Madison, Wis.). Briefly, cells were transiently transfected with APP751 or vehicle for 48 h and then switched into selection medium containing a concentration range of the antibiotic 0418 (100-600 μg/mL) to determine the optimal dose for stable selection. Selection media was changed every 3 days to remove non-viable cells. After 3 weeks, 200 μg/mL 0418 yielded distinct colonies while all vehicle-transfected cells were non-viable. Cells were maintained in 10% FBS media containing 200 μg/mL 0418 for expansion.

Cell Viability Assay:

RA-SY5Y, at 2×10⁴ cells/well, were seeded in 96-well microplates and treated for 48 h with a range of concentrations of MβCD, DMSO, ceramide, cholesterol or oleic acid to determine effective doses for 50-70% viability (data not shown), or in the presence of a range of concentrations of Leptin to determine effective doses to prevent cell death. Viability was assessed using the Cell-Titer Blue Viability Assay (Promega) by adding 20 μL of the reagent to each well for 4 h, and plates read by a microplate reader with fluorescence capabilities at Ex_{530-570 nm/}Em_{580-620 nm}. Viability was determined using a standard curve of known cell number and plotted as a percent of non-treated or vehicle control.

### Preparation of Cell Lysates:

RA-SY5Y were treated with Leptin (100 ng/ml) in the presence of ceramide (25 μM), cholesterol (27.5 μg/mL), oleic acid (30 μg/mL) or vehicle (MβCD—675 μg/mL; or DMSO—0.125%) for 6 h, and then harvested by scraping. Preparation of whole cell lysates from cell pellets was performed as described previously (32).

AMPK Activity Assay:

AMPK activity in RA-SY5Y cell lysates was determined using the CycLex AMPK Kinase Assay Kit (MBL International; Woburn, Mass.), as previously described (33, 34). Briefly, “relative AMPK activity”, hereafter referred to as “AMPK activity”, is defined as Compound C-sensitive protein kinase activity in cell lysates. Titration of various Compound C doses identified 10 μM as the dose in which there was no further reduction in kinase activity upon increasing concentration (data not shown). Lysates were incubated in the presence or absence of 10 μM Compound C, and protein kinase activity determined by measuring phosphorylation of the Insulin Receptor Substrate-I (IRS-1) through immunoassay and conversion of a chromogenic substrate at an absorbance of 450 nm (Ai50). Normalized AMPK activity was defined as: [(A450_{lysate}−A450_{lysate+Compound C})/μg protein_{lysate}]×10³.

SIRT Activity Assay:

“Total sirtuin”, hereafter termed “SIRT’, activity in cell lysates was determined using the HDAC Fluorimetric Cellular Activity Assay Kit (Enzo Life Sciences; Plymouth Meeting, Pa.), according to manufacturer's specified guidelines. Briefly, SIRT activity is defined as nicotinamide-sensitive deacetylase (class III HDAC) activity in cell lysates. 5 mM nicotinamide was identified by the manufacturer as a dose in which there was no further reduction in deacetylase activity upon increasing concentration. RA-SY5Y cell lysates were incubated in the presence or absence of 5 mM nicotinamide, and SIRT activity determined by adding the Flour de Lys Substrate for deacetylation followed by exposure to the Fluor de Lys Developer to generate a fluorescent signal for detection using a fluorimeter (EX_{-350-380 nm}/Em_{440-460 nm}). Normalized SIRT activity was measured in units of fluorescence intensity (Fi) and defined as: (Fi_{lysate}−Fi_{lysate+nicotinamide})/μg protein_{lysate}.

ELISAs:

Aβ₍₁₋₄₀₎ levels in cell culture media from SY5Y_{APP751} cells treated for 18 h with the aforementioned metabolic insults in the presence or absence of Leptin, were determined using the Human βAmyloid 1-40 ELISA kit (Invitrogen; Carlsbad, Calif.), and phospho- and total tau levels in RASY5Y lysates were determined using the Human Tau pSer396, pThr231 and Total Tau ELISA kits (Invitrogen) according to manufacturer's specific instructions. Aβ₍₁₋₄₀₎, phosho- and total tau levels were calculated from a standard curve developed with OD at 450 nm using 8 serial dilutions of known concentration.

Statistical Analyses:

Statistical data analyses were performed with analysis of variance and Tukey-Kramer multiple comparisons test. p<0.05 was considered statistically significant.

### Results

Effect of Metabolic Challenges on Neuronal Cell Viability:

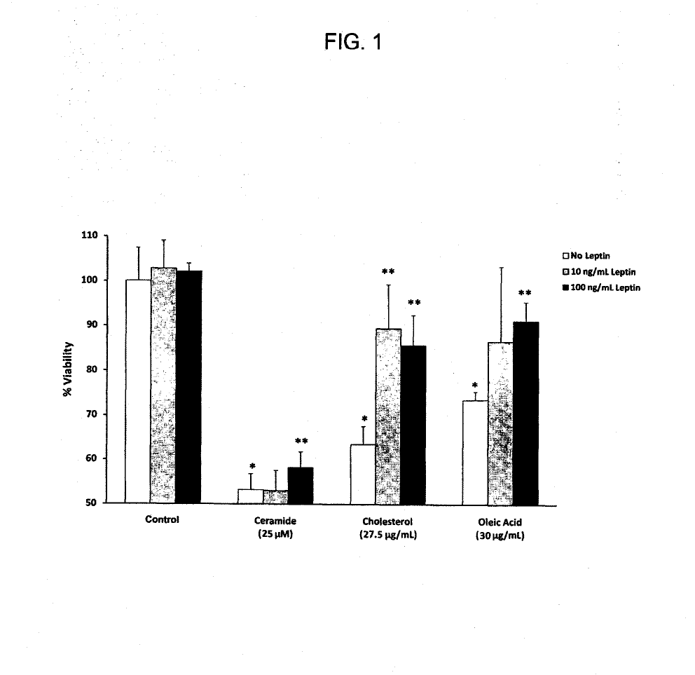

Doses of the lipids which decreased neuronal viability by 25-50% (Ceramide—25 μM; Cholesterol—27.5 μg/mL and Oleic Acid—30 μg/mL) after 18 h treatment (data not shown) were used to determine whether co-treatment with Leptin, an important modulator of lipid homeostasis, could attenuate the toxic effects of the insults (FIG. 1). RA-SY 5Y were treated for 18 h with either a low (10 ng/mL; gray bars) or moderate (100 ng/mL; black bars) dose of Leptin or control (white bars) in the presence or absence of ceramide, cholesterol or oleic acid, and cell viability measured. All lipid insults induced a significant (p<0.05) decrease in cell viability in the range of 35±15% when treated with control alone. Low dose Leptin was able to significantly (p<0.05) improve viability only in response to the cholesterol insult, while a moderate dose significantly (p<0.05) improved viability in response to all challenges. A third treatment group utilizing a high dose (1000 ng/mL) of Leptin was not significantly (p>0.05) different from the moderate group's viability (data not shown). Of all the lipid insults, Leptin most significantly attenuated the toxic effects of cholesterol (second group from right).

Leptin Increases Cellular Metabolism in Response to Metabolic Challenges:

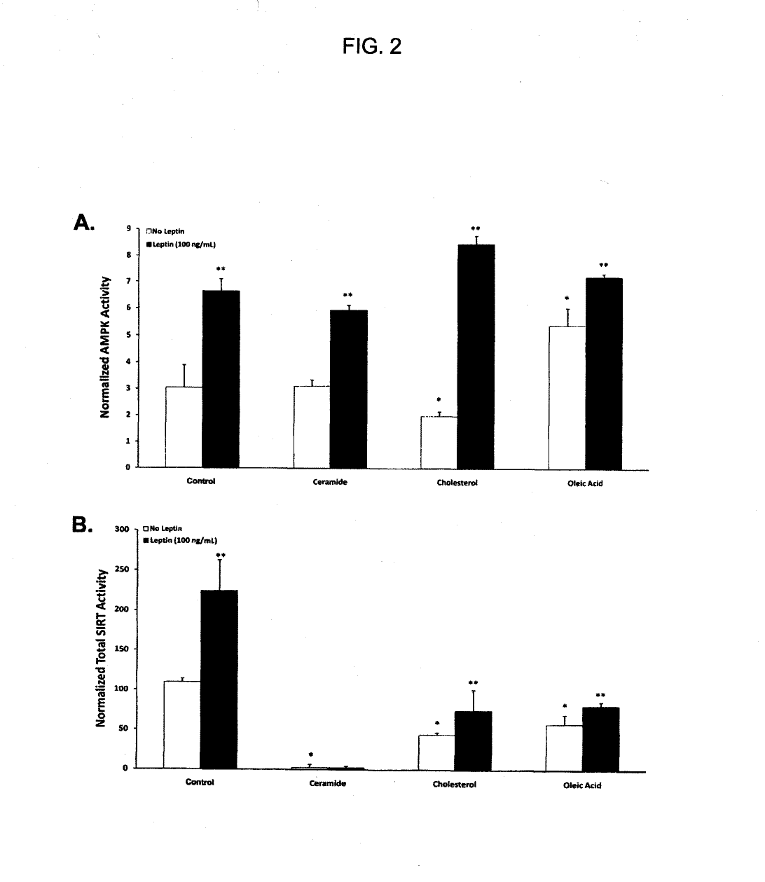

We have previously shown that Leptin regulates AD pathways via activation of the cellular energy sensors, AMP-activated protein kinase (AMPK) and the sirtuins (SIRT) in neuronal cells (35, 36). To this end, we determined whether Leptin has the ability to attenuate these effects on cellular energetics in an acute model of metabolic stress (FIG. 2). Utilizing similar lipid doses as for the viability experiments, RA-SY5Y were treated for 6 h with Leptin (100 ng/mL) or vehicle in the presence or absence of ceramide, cholesterol or oleic acid, and cellular energy status measured by AMPK (FIG. 2A) or SIRT activity (FIG. 2B). All insults were able to significantly (p<0.05) suppress SIRT activity (FIG. 2B; white bars), while only cholesterol showed a similar effect on AMPK activity at the specified dose (FIG. 2A; second white bar from right). Treatment with Leptin (FIGS. 2A and B; gray bars) was able to significantly (p<0.05) boost both AMPK and SIRT activity in response to all lipid insults, with the exception of ceramide for SIRT (FIG. 2B; second gray bar from left). Interestingly, as was observed for the viability experiments, Leptin most significantly attenuated the suppressive effects of cholesterol (second group from right) on cellular energy metabolism.

Lectin Suppresses AD-Related Pathway Activation Following Metabolic Challenges:

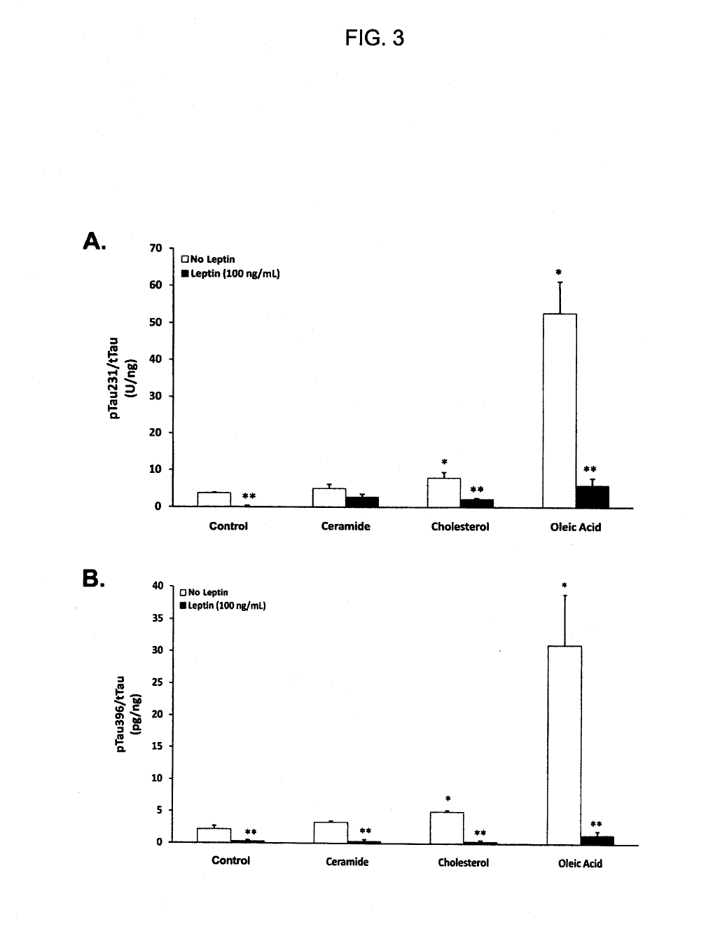

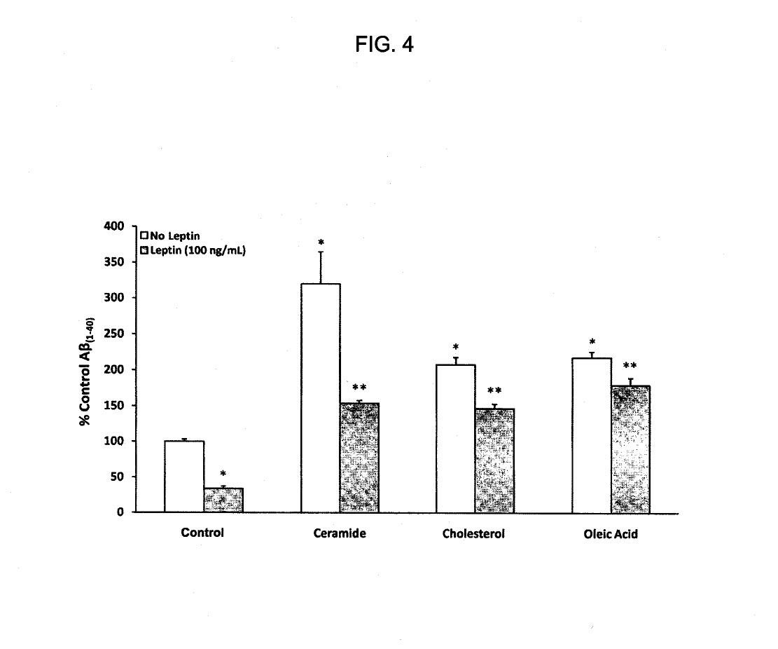

Metabolic stress has been linked to the activation of AD pathological pathways, with lipids known to play an important role (21-23). We therefore investigated the extent to which lipids can induce hyperphosphorylation of tau (FIG. 3) and AP production (FIG. 4) in neuronal cells, and determined whether Leptin could prevent these effects. Utilizing similar lipid doses as previous experiments, RA-SY5Y were treated for 6 h with Leptin (100 ng/mL) or control in the presence or absence of ceramide, cholesterol or oleic acid, and phosphorylation of tau at two different epitopes, pTau231 (FIG. 3A) or pTau396 (FIG. 3B), as well as total tau measured by ELISA. Challenging cells with either cholesterol (second group from right) or oleic acid (far right group) induced significant (p<0.05) increases in tau phosphorylation at either epitope, with oleic acid incurring the greatest effect (approximately 15 to 20-fold increase). Simultaneous treatment with Leptin significantly (p<0.05) prevented this hyperphosphorylation (far right group, gray bars) more dramatically than the other insults.

In parallel to the tau studies we investigated if our metabolic challenge model could exacerbate the extracellular production of AP, and likewise determined whether Leptin could prevent these effects (FIG. 4). SY5Y stably over-expressing human APP751 were treated for 18 h with Leptin (100 ng/mL) or control in the presence or absence of ceramide, cholesterol or oleic acid, and the amount of extracellular APo4 o) measured by ELISA. All lipid insults induced a significant (p<0.05) increase in the amount of Aβ₍₁₋₄₀₎ produced (white bars), with ceramide (second group from left, white bar) incurring the greatest effect (−3-fold increase). Treatment with Leptin (gray bars) significantly (p<0.05) reduced Aβ₍₁₋₄₀₎ levels as similar to control (first group from right, white bar) for all insults. Leptin's ability to reduce Aβ₍₁₋₄₀₎ production following ceramide challenge (second group from left, gray bar) was the most dramatic of all insults.

### Discussion

Excess buildup of lipids exerts toxic effects on the brain (37). Lipids are a broad group of molecules which encompass fatty acids, glycero-, phospho-, sphingo- and sterol lipids, among others. Our studies began by investigating the relationship between several types of lipids and viability of SY5Y neuroblastoma cells differentiated with retinoic acid (RA-SY5Y). We specifically utilized: a) Ceramide, a lipid composed of sphingosine and a fatty acid, typically found in cell membranes, b) Cholesterol, a waxy steroid of fat, also found in cell membranes; and c) Oleic Acid, a monounsaturated omega-9 fatty acid found in vegetable and animal fats.

Prolonged states of positive energy balance due to high levels of fatty acids, triglycerides and/or cholesterol, as is often the case in obesity, can disrupt cellular energy metabolism and may have similar negative effects in neurons (38). Metabolic diseases, particularly obesity and diabetes, have been associated with increased risk of cognitive impairment and AD (38). In support, the contribution of diet and nutrition to AD incidence has been extensively documented (1, 2). Leptin, an adipocyte-derived hormone which physiologically functions in the control of lipid storage and mobilization, impedes production of Aβ and hyperphosphorylation of tau (30-32). These epidemiological findings combined with Leptin's ability to regulate both lipid metabolism and AD pathobiology led us to investigate the relationship between lipids, Leptin and activation of AD-related pathways.

We utilized three classes of lipids with known effects on AD pathways to serve as metabolic challenges in testing Leptin's protective functions. We first determined the optimal dose that would decrease neuronal viability by 25-50% for each insult. We surmised that doses of these lipids which induce a modest degree of cell death should also activate AD pathways and potentially impede cellular metabolism. This presumption was predominantly valid in that the insults significantly increased tau phosphorylation (FIG. 3) and extracellular production of Aβ (FIG. 4), and depressed cellular metabolism through deactivation of the energy sensors, AMPK and SIRT (FIG. 2).

For most lipid insults, Leptin co-administration negated the deleterious effects; however, each assay identified a unique insult that Leptin was most able to abrogate. For viability and cellular metabolism studies Leptin was most successful in preventing the effects of cholesterol, while for tau and Aβ Leptin was most successful in preventing the effects of oleic acid and ceramide, respectively. This indicates that heterologous deleterious metabolic challenges on neurons may be responsible in preferentially triggering either the amyloidogenic or tau pathways. However, most importantly, Leptin can correct the metabolic imbalances that high concentration of extracellular lipids may induce to neurons preventing AD-like cascades.

These results have been the core of our novel cell-based screening platform that utilizes an array of assay-specific lipid challenges to identify both novel and existing compounds which behave similar to Leptin in modulating viability, energy metabolism and AD pathways. The platform utilizes cell viability as the preliminary screen to demonstrate feasibility as well as determine the effective dose for the subsequent readouts of the assay. Discovery of lead compounds will initiate testing in animal models of AD, and further drug development efforts will determine therapeutic significance.

Some of the categories that we are exploring include: a) High-affinity activators of the Leptin receptor, which could entail Leptin-like peptides, such as muteins or fusion proteins, or small molecule Leptin receptor agonists; b) Modulators of components of the Leptin signaling pathway; c) Modulators of AMPK and SIRT (35, 36); d) Novel insulin-like or insulin-sensitizing compounds, such as the PPARγ agonist thioglitazones; e) Inhibitors to factors involved in lipid biosynthesis, such as sterol regulatory-element binding proteins (SREBPs).

Attenuation of the detrimental effects that exposure of neurons to excess lipid levels imparts is clinically significant. The progressive deterioration of brain lipid homeostasis in AD patients can elicit locally increased cholesterol (39), ceramide (26) and oleic acid (40) levels, further suggesting that assessment of the molecular and cell biology of lipids in the context of Alzheimer's disease and the impact of lipid-related changes on neurometabolism should be of interest to basic and applied scientists alike (41). Our present report provides evidence that Leptin can suppress activation of AD pathological pathways in response to high doses of these lipids. These findings further support Leptin as a potential AD therapeutic and provide a framework to screen novel and existing compounds which act similar or complementary to Leptin.

### REFERENCES

1. Bhat, N. R. (2010) Linking cardiometabolic disorders to sporadic Alzheimer's disease: a perspective on potential mechanisms and mediators. Journal of neurochemistry 115, 551-562.
2. Pasinetti, G. M., and Eberstein, J. A. (2008) Metabolic syndrome and the role of dietary lifestyles in Alzheimer's disease. Journal of neurochemistry 106, 1503-1514.
3. Fishel, M. A., Watson, G. S., Montine, T. J., Wang, Q., Green, P. S., Kulstad, J. J., Cook, D. G., Peskind, E. R., Baker, L. D., Goldgaber, D., Nie, W., Asthana, S., Plymate, S. R., Schwartz, M. W., and Craft, S. (2005) Hyperinsulinemia provokes synchronous increases in central inflammation and beta-amyloid in normal adults. Arch Neural 62, 1539-1544.
4. Hooijmans, C. R., Van der Zee, C. E., Dederen, P. J., Brouwer, K. M., Reijmer, Y. D., van Groen, T., Broersen, L. M., Lutjohann, D., Heerschap, A., and Kiliaan, A. I. (2009) DHA and cholesterol containing diets influence Alzheimer-like pathology, cognition and cerebral vasculature in APPswe/PS 1 dE9 mice. Neurobiology of disease 33, 482-498.
5. Whitmer, R. A., Gunderson, E. P., Barrett-Connor, E., Quesenberry, C. P., Jr., and Yaffe, K. (2005) Obesity in middle age and future risk of dementia: a 27 year longitudinal population based study. Bmj 330, 1360.
6. Whitmer, R. A., Gustafson, D. R., Barrett-Connor, E., Haan, M. N., Gunderson, E. P., and Yaffe, K. (2008) Central obesity and increased risk of dementia more than three decades later. Neurology 71, 1057-1064.
7. Huang, X. F., Koutcherov, I., Lin, S., Wang, H. Q., and Storlien, L. (1996) Localization of leptin receptor mRNA expression in mouse brain. Neuroreport 7, 2635-2638.
8. Harvey, J., Shanley, L. J., O'Malley, D., and Irving, A. J. (2005) Leptin: a potential cognitive enhancer? Biochem Soc Trans 33, 1029-1032.
9. Dietrich, M. O., Spuch, C., Antequera, D., Rodal, I., de Yebenes, J. G., Molina, J. A., Bermejo, F., and Carro, E. (2008) Megalin mediates the transport of leptin across the blood-CSF barrier. Neurobiology of aging 29, 902-912.
10. Adam, C. L., and Findlay, P. A. (2010) Decreased blood-brain leptin transfer in an ovine model of obesity and weight loss: resolving the cause of leptin resistance. Int J Obes (Lond) 34, 980-988.
11. Barrett-Connor, E., Edelstein, S. L., Corey-Bloom, J., and Wiederholt, W. C. (1996) Weight loss precedes dementia in community-dwelling older adults. J Am Geriatr Soc 44, 1147-1152.
12. Mazzali, G., Bissoli, L., Gambina, S., Residori, L., Pagliari, P., Guariento, S., Sun, M., Broggio, E., Bosello, 0., and Zamboni, M. (2002) Energy balance in Alzheimer's disease. J Nutr Health Aging 6, 247-253.
13. Olsson, T., Nasman, B., Rasmuson, S., and Ahren, B. (1998) Dual relation between leptin and cortisol in humans is disturbed in Alzheimer's disease. Biol Psychiatry 44, 374-376.
14. Power, D. A., Noel, J., Collins, R., and O'Neill, D. (2001) Circulating leptin levels and weight loss in Alzheimer's disease patients. Dement Geriatr Cogn Disord 12, 167-170.
15. Zhou, R., Deng, J., Zhang, M., Zhou, H. D., and Wang, Y. J. (2011) Association between bone mineral density and the risk of Alzheimer's disease. Journal of Alzheimer's disease: JAD 24, 101-108.
16. Bigalke, B., Schreitmuller, B., Sopova, K., Paul, A., Stransky, E., Gawaz, M., Stellos, K., and Laske, C. (2011) Adipocytokines and CD34 Progenitor Cells in Alzheimer's Disease. PloS one 6, e20286.
17. Ray, S., and Wyss-Coray, A. (2005) Methods and Compositions for Diagnosis,

Stratification, and monitoring of Alzheimer's Disease and other neurological disorders in body fluids. In WO 20051052592 A2, Satoris, Inc.
18. Holden, K. F., Lindquist, K., Rosano, C., Tylaysky, F. A., Harris, T. B., and Yaffe, K. (2006) Low Serum Leptin is Associated with Poor Cognitive Performance in the Elderly. In American Academy of Neurology, 58th Annual Meeting p. 541.006, San Diego.
19. Holden, K. F., Lindquist, K., Tylaysky, F. A., Rosano, C., Harris, T. B., and Yaffe, K. (2008) Serum leptin level and cognition in the elderly: Findings from the Health ABC Study. Neurobiol Aging.
20. Lieb, W., Beiser, A. S., Vasan, R. S., Tan, Z. S., Au, R., Harris, T. B., Roubenoff, R., Auerbach, S., DeCarli, C., Wolf, P. A., and Seshadri, S. (2009) Association of plasma leptin levels with incident Alzheimer disease and MRI measures of brain aging. JAMA: the journal of the American Medical Association 302, 2565-2572.
21. Puglielli, L., Tanzi, R. E., and Kovacs, D. M. (2003) Alzheimer's disease: the cholesterol connection. Nat Neurosci 6, 345-351.
22. Glockner, F., Meske, V., Lutjohann, D., and Ohm, T. G. (2011) Dietary cholesterol and its effect on tau protein: a study in apolipoprotein E-deficient and P301L human tau mice. Journal of neuropathology and experimental neurology 70, 292-301.
23. Wilson, D. M., and Binder, L. I. (1997) Free fatty acids stimulate the polymerization of tau and amyloid beta peptides. In vitro evidence for a common effector of pathogenesis in Alzheimer's disease. The American journal of pathology 150, 2181-2195.
24. Hooff, G. P., Peters, I., Wood, W. G., Muller, W. E., and Eckert, G. P. (2010) Modulation of cholesterol, farnesylpyrophosphate, and geranylgeranylpyrophosphate in neuroblastoma SH-SY5Y-APP695 cells: impact on amyloid beta-protein production. Mal Neurobiol 41, 341-350.
25. Darios, F., Muriel, M. P., Khondiker, M. E., Brice, A., and Ruberg, M. (2005) Neurotoxic calcium transfer from endoplasmic reticulum to mitochondria is regulated by cyclindependent kinase 5-dependent phosphorylation of tau. The Journal of neuroscience: the official journal of the Society for Neuroscience 25, 4159-4168.
26. Haughey, N. J., Bandaru, V. V., Bae, M., and Mattson, M. P. (2010) Roles for dysfunctional sphingolipid metabolism in Alzheimer's disease neuropathogenesis. Biochimica et biophysica acta 1801, 878-886.
27. Liu, Y., Yang, L., Conde-Knape, K., Beher, D., Shearman, M. S., and Shachter, N. S. (2004) Fatty acids increase presenilin-1 levels and [gamma]-secretase activity in PSwt-1 cells. Journal of lipid research 45, 2368-2376.
28. Merched, A., Xia, Y., Visvikis, S., Serot, J. M., and Siest, G. (2000) Decreased high density lipoprotein cholesterol and serum apolipoprotein AI concentrations are highly correlated with the severity of Alzheimer's disease. Neurobiology of aging 21, 27-30.
29. Mahley, R. W., and Huang, Y. (2006) Apolipoprotein (apo) E4 and Alzheimer's disease: unique conformational and biophysical properties of apoE4 can modulate neuropathology. Acta Neural Scand Suppl 185, 8-14.
30. Fewlass, D. C., Noboa, K., Pi-Sunyer, F. X., Johnston, J. M., Yan, S. D., and Tezapsidis, N. (2004) Obesity-related leptin regulates Alzheimer's Abeta. Faseb J 18, 1870-1878.
31. Greco, S. J., Bryan, K. J., Sarkar, S., Zhu, X., Smith, M. A., Ashford, J. W., Johnston, J. M., Tezapsidis, N., and Casadesus, G. (2010) Leptin reduces pathology and improves memory in a transgenic mouse model of Alzheimer's disease. Journal of Alzheimer's disease: JAD 19, 1155-1167.
32. Greco, S. J., Sarkar, S., Johnston, J. M., Zhu, X., Su, B., Casadesus, G., Ashford, J. W., Smith, M. A., and Tezapsidis, N. (2008) Leptin reduces Alzheimer's disease-related tau phosphorylation in neuronal cells. Biochem Biophys Res Commun 376, 536-541.
33. Wang, Y., Nishi, M., Doi, A., Shono, T., Furukawa, Y., Shimada, T., Furuta, H., Sasaki, H., and Nanjo, K. (2010) Ghrelin inhibits insulin secretion through the AMPK-UCP2 pathway in beta cells. FEES letters 584, 1503-1508.
34. Yeh, C. H., Chen, T. P., Wang, Y. C., Lin, Y. M., and Fang, S. W. (2010) AMP-activated protein kinase activation during cardioplegia-induced hypoxia/reoxygenation Injury attenuates cardiomyocytic apoptosis via reduction of endoplasmic reticulum stress. Mediators Jnflamm 2010, 130636.
35. Greco, S. J., Sarkar, S., Johnston, J. M., and Tezapsidis, N. (2009) Leptin regulates tau phosphorylation and amyloid through AMPK in neuronal cells. Biochem Biophys Res Commun 380, 98-104.
36. Greco, S. J., Hamzelou, A., Johnston, J. M., Smith, M. A., Ashford, J. W., and Tezapsidis, N. (2011) Leptin boosts cellular metabolism by activating AMPK and the sirtuins to reduce tau phosphorylation and beta-amyloid in neurons. Biochemical and biophysical research communications 414, 170-174.
37. Martins, I. C., Kuperstein, I., Wilkinson, H., Maes, E., Vanbrabant, M., Jonckheere, W., Van Gelder, P., Hartmann, D., D'Hooge, R., De Strooper, B., Schymkowitz, J., and Rousseau, F. (2008) Lipids revert inert Abeta amyloid fibrils to neurotoxic protofibrils that affect learning in mice. The EMBO journal 27, 224-233.
38. Kapogiannis, D., and Mattson, M. P. Disrupted energy metabolism and neuronal circuit dysfunction in cognitive impairment and Alzheimer's disease. Lancet Neurol 10, 187-198.
39. Leduc, V., Jasmin-Belanger, S., and Poirier, J. (2010) APOE and cholesterol homeostasis in Alzheimer's disease. Trends in molecular medicine 16, 469-477.
40. Fraser, T., Tayler, H., and Love, S. (2010) Fatty acid composition of frontal, temporal and parietal neocortex in the normal human brain and in Alzheimer's disease. Neurochemical research 35, 503-513.
41. Foley, P. Lipids in Alzheimer's disease: A century-old story. Biochim Biophys Acta 1801, 750-753.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for rescuing neurons from Alzheimer's Disease related pathways triggered by lipid burden or metabolic insult, the method comprising contacting the neuronal cell population with a composition containing an effective amount of Leptin, a Leptin analog or derivative, or a Leptin agonist, and a carrier, wherein the effective amount of Leptin, the Leptin analog or derivative, or the Leptin agonist is effective to rescue neurons from Alzheimer's Disease related pathways triggered by the lipid burden or metabolic insult.

2. The method of claim 1, wherein the composition inhibits decreases in cell viability in a neuronal cell population that are triggered by lipid burden or metabolic insult.

3. The method of claim 1, wherein the composition inhibits decreases in enzymatic activity of at least one family member of Sirtuins (SIRT) in a neuronal cell population that are triggered by the lipid burden or metabolic insult.

4. The method of claim 1, wherein the composition decreases the phosphorylation of tau at sites known to be hyperphosphorylated in AD in a neuronal cell population that are triggered by the lipid burden or metabolic insult.

5. The method of claim 1, wherein the composition decreases the accumulation of Aβ in a neuronal cell population that are triggered by the lipid burden or metabolic insult.

6. An in vitro assay method to screen for compounds that prevent the activation of AD-related pathways that are mediated by lipid burden or metabolic insult.
